(19) United States
(12) Patent Application Publication
(10) Pub. No.: US 2009/0209637 A1
(43) Pub. Date: Aug. 20, 2009

(54) USE OF CHROMEN-4-ONE DERIVATIVES

(76) Inventors: Christophe Carola, Langen (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/439,340

(22) PCT Filed: Aug. 30, 2006
(86) PCT No.: PCT/EP2006/008474

§ 371 Date: Feb. 27, 2009

(51) Int. Cl. A61K 31/352; C07D 311/22

(52) U.S. Cl. 514/456; 549/401

(57) ABSTRACT

The invention relates to the use of chromen-4-one derivatives of the formula I

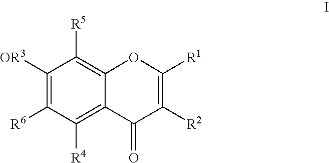

where R¹ and R² may be identical or different and are selected from H, —C(═O)—R⁷, —C(═O)—OR⁷, alkyl groups, alkenyl groups, hydroxyalkyl groups and/or cycloalkyl groups and/or cycloalkenyl groups, R³ is H or alkyl groups, R⁴ is H or OR⁸, R⁵ and R⁶ are selected from —H, —OH, alkyl groups, alkenyl groups and hydroxyalkyl groups, and R⁷ is H, alkyl groups, a polyhydroxyl compound, such as, preferably, an ascorbic acid radical or glycosidic radicals, and R⁸ is H or alkyl groups, where at least two of the substituents R¹, R² and R⁴-R⁶ are different from H or at least one substituent from R¹ and R² is —C(═O)—R⁷ or —C(═O)—OR⁷, to prevent, reduce or combat signs of cellulite and/or reduce localized fatty excesses.

The present invention relates to the use of chromen-4-one derivatives to prevent, reduce or combat signs of cellulite and/or reduce localized fatty excesses.

Cellulite is a term applied to a skin condition associated with the lumps, bumps and dimples that appear on the thighs of many women. Cellulite primarily afflicts the thighs and buttocks but may also be present on the stomach and upper arms. This condition is frequently described as “orange peel skin”, “mattress phenomena” or the “cottage cheese effect”. Cellulite afflictions are a stubborn problem causing emotional and psychological distress to many women. Although the etiology of cellulite is poorly understood, the main etiological factor appears to be local accumulation of fat in a regional compartment.

It has been proposed that the anatomical structure of subcutaneous adipose tissue is the major cause of cellulite. The histological studies of subcutaneous tissues from men and women suggest that the fat lobules are larger and more vertical in women than men. As a result, these larger, less restricted lobules can express outward against the dermis causing the bumps and dimples characteristic of cellulite. The femoral subcutaneous fat deposits in women also tend to be more lipogenic and less lipolytic than abdominal subcutaneous or visceral fat due to the difference in the distribution of alpha and beta adrenergic receptors on adipocytes in these different regions. Increased lipolysis or fat reduction of these selected subcutaneous adipose sites may lead to a reduction or the prevention of cellulite.

Among the methods for stimulating lipolysis, the most commonly known and used is that which consists in inhibiting the phosphodiesterase in order to prevent or at least limit the rate of degradation of cyclic AMP. In effect, the phosphodiesterase destroys cyclic AMP by transforming it into 5′ AMP so that it cannot function as a lipolysis activator. Topical application for the treatment of cellulite of agents capable of distributing or reducing local fat accumulation by lipolytic action thereby improving the aesthetic appearance of the skin has been used. Among the common agents for treatment of cellulite as slimming agents are xanthine analogs such as theobromine, aminophylline, caffeine or theophylline. These agents block the antilipolytic action of adenosine, a potent endogenous inhibitor of lipolysis.

Other known methods in lipolysis stimulation are achieved by inhibiting phosphodiesterase in order to prevent or at least limit the degradation of cAMP. Xanthine based adenosine antagonists such as caffeine or theophylline are also known to be effective phosphodiesterase inhibitors.

Other existing methods for the treatment of cellulite have been the stimulation of adenylate cyclase to increase cAMP levels (beta-adrenergic agonists) or to block the antilipolytic inactivation of adenylate cyclase (alpha-2-adrenergic antagonists). Greenway et al. (U.S. Pat. No. 4,588,724) disclose that isoproterenol, a known beta agonist (beta-adrenergic stimulator), is effective for the treatment of cellulite by stimulating lipolysis. Greenway et al. (U.S. Pat. Nos. 4,588,724 and 4,525,359) disclose that creams based on yohimbine, a known alpha-2-blocker applied to women's skin showed a decrease in thigh circumference. Soudant et al. (U.S. Pat. No. 5,194,259) disclose a Ginkgo biloba, a known alpha-2-blocker, as a lipolytic agent in combination with at least one other alpha-2-blocker in a slimming cosmetic composition.

Moreover, it has also been known to use certain oleosoluble vegetable extracts which, according to a different mechanism, can also act as a slimming agent. For instance, in U.S. Pat. No. 4,795,638 there is disclosed a thermo slimming cosmetic composition containing an oil-soluble plant extract having slimming action. Representative of these oil-soluble plant extracts are vegetable extracts including, principally, those of climbing ivy (Hedera helix), arnica (Arnica montana), rosemary (Rosmarinus officinalis N), marigold (Calendula officinalis), sage (Salvia officinalis N), ginseng (Panax ginseng), St. Johns-wart (Hypericum perforatum), ruscus (Ruscus aculeatus), meadowsweet (Filipendula ulmaria L) and orthosiphon (Ortosifon stamincus Benth), as well as mixtures of these vegetable extracts.

Accordingly, it is an object of the present invention to provide methods for reducing or preventing cellulite in mammalian skin.

Retinoids reduce the signs of cellulite when applied topically to human skin, particularly female skin (EP-A-866 693; U.S. Pat. No. 5,051,449). Mattressing is partially effaced and the skin contour becomes more even. Lumpy-bumpy skin becomes smoother. Topical application may be performed by a number of methods, which will be apparent to one skilled in the art of pharmacology. In one embodiment of the present invention, the retinoid is applied to skin affected by cellulite by injunction or any conventional topical applicator device known to those skilled in the art of pharmacology.

Owing to the constantly increasing demand for active ingredients for the preventative treatment of human skin and human hair against ageing processes and harmful environmental influences, the object of the present invention was to provide alternative active ingredients which exhibit the effects already mentioned at the outset, are sufficiently oxidation- and photostable and can readily be formulated. The compositions prepared therewith should furthermore have as far as possible a low irritation potential for the skin, as far as possible have a positive influence on water binding in the skin, retain or increase skin elasticity and thus promote smoothing of the skin. In addition, they should preferably create a pleasant skin feeling on application to the skin.

Surprisingly, it has now been found that certain chromen-4-one derivatives (chromone derivatives) are suitable as active ingredients having the profile described.

The present invention relates firstly to the use of at least one compound of the formula I

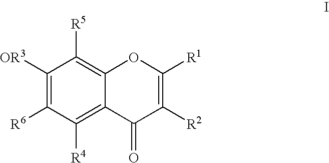

or of a composition comprising at least one compound of the formula I, where
R¹ and R² may be identical or different and are selected from
   H, —C(═O)—R⁷ and —C(═O)—OR⁷,
   straight-chain or branched C₁- to C₂₀-alkyl groups,
   straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
      C₃- to C₁₀-cycloalkyl groups and/or C₃- to C₁₂-cycloalkenyl groups,
         where the rings may each also be bridged by —(CH₂)ₙ— groups, where n=1 to 3,
R³ is H or straight-chain or branched C₁- to C₂₀-alkyl groups,
R⁴ is H or OR⁸,
R⁵ and R⁶ may be identical or different and are selected from —H and —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ is H, straight-chain or branched C₁- to C₂₀-alkyl groups, a polyhydroxyl compound, such as, preferably, an ascorbic acid radical or glycosidic radicals, and
R⁸ is H or straight-chain or branched C₁- to C₂₀-alkyl groups,
   where at least two of the substituents R¹, R² and R⁴-R⁶ are different from H or at least one substituent from R¹ and R² is —C(═O)—R⁷ or —C(═O)—OR⁷, to prevent, reduce or combat signs of cellulite and/or reduce localized fatty excesses.

The use of claim 1 includes the non-therapeutic use. The use of claim 13 includes the therapeutic use.

For the purposes of the present invention, the term “compound of the formula I” basically also includes the salts of the compounds of the formula I. The preferred salts here include, in particular, alkali metal and alkaline earth metal salts as well as ammonium salts, but in particular sodium and potassium salts.

Compounds of formula I and structurally related compounds are known from the literature, partially also for cosmetic applications:

The use of certain 2-(alkyl)carboxyl- or 2-(alkyl)phenyl-substituted chromen-4-one derivatives in combination with divalent zinc in pharmaceutical and cosmetic compositions is disclosed in EP-A-0 304 802. The compositions are suitable for the treatment of skin, in particular for the treatment of dermatoses, including atopic eczema.

EP-A-0 424 444 discloses the use of salts of chromonecarboxylic acid in cosmetics for combating skin ageing. The compound exhibits a UV-filtering action here and has the following effects in animal experiments: the proportion of bound lipids in the skin increases, the proportion of soluble collagen in the skin is increased, the resistance of the skin to the effects of the fibroplatic proteases collagenase and elastase is increased.

U.S. Pat. No. 6,019,992 discloses cosmetic compositions which comprise 4-chromanone and are suitable for the treatment of aged, dry or wrinkled skin. It is shown here that 4-chromanone promotes cell differentiation and stimulates lipid production in keratinocyte cultures.

EP-A-1 216 692 discloses the use of 2-methyl-2-(β-carboxyethyl)chroman derivatives in cosmetic compositions. The said compositions are particularly suitable for prophylaxis against ageing processes of skin and hair and for prophylaxis against dry skin, wrinkle formation and pigment defects.

Compositions for topical application which comprise chromone derivatives, such as, for example, chromone, 7-hydroxychromone, 7-methoxychromone, 5,7-dihydroxy-2-methylchromone, 3-methyl-2-butenyloxychromone, 3-acetyl-5,7-dihydroxy-2-methylchromone, 5-hydroxychromone, n-pentyl 7-methoxychromone-2-carboxylate, n-undecyl 5-methoxychromone-2-carboxylate, 5-hydroxy-7-methoxy-2-methylchromone, 7-methoxychromone-2-carboxylic acid, n-pentylchromone-2-carboxylic acid, 5-methoxychromone and chromone-2-carboxylic acid, are disclosed in Japanese patent application JP 05/301813. The chromone derivatives act as skin-tolerated tyrosinase inhibitors which reduce hyperpigmentation of the skin.

Japanese patent application JP 09/188608 discloses the use of substituted chromone derivatives, such as, in particular, 5,7-dihydroxychromones, 7-methoxychromones, 5-hydroxy-7-methoxy-2-methylchromone and 5-hydroxy-2-methylchromone, as active ingredient against grey hair. The action here is attributed to activation of the coloured pigment-forming cells and the increase in melanogenesis.

A composition against skin ageing comprising chromone derivatives which are substituted in the 2-position by C₁₋₁₅-alkyl and have H, OH or alkoxy substitution in the 7-position, in combination with aminopropanol derivatives is disclosed in JP 10/194919.

Cosmetic compositions which comprise substituted chromone derivatives, such as, for example, 2-(1-ethylpentyl)chromone, 5,7-dihydroxychromones, 7-methoxychromones, 5-hydroxy-7-methoxy-2-methylchromone and 5-hydroxy-2-methylchromone, and aromatic compounds having a melting point of —10° C. or above are disclosed in JP 10/114640. The chromone derivative here simplifies incorporation of the aromatic compound into the cosmetic formulation.

The use of compounds of formula I in cosmetics, as agents for the care, preservation or improvement of the general state of the skin or hair, for prophylaxis against time- and/or light-induced ageing processes of the human skin or human hair, in particular for prophylaxis against dry skin, wrinkle formation and/or pigment defects, and/or for the reduction or prevention of the harmful effects of UV rays on the skin, and for prophylaxis against or reduction of skin unevenness, such as wrinkles, fine lines, rough skin or large-pored skin, and for the prophylaxis and/or prevention of premature skin ageing, in particular for the prophylaxis and/or prevention of light- or ageing-induced wrinkling of the skin, for the reduction of pigmentation and keratosis actinica, and for the prophylaxis and/or treatment of all diseases which are associated with normal skin ageing or light-induced ageing of the skin is described in EP-A-1 508 327.

Now, it was found that compounds of formula I can prevent, reduce or combat signs of cellulite and/or reduce localized fatty excesses. Furthermore the compounds can be used as active agent in the prevention or treatment of cellulite.

While the inventor does not wish to be bound by any particular theory, it is believed that compounds of formula I alleviate cellulite, particularly the mattressing phenomenon, through a combination of factors, including:
(1) Stimulating fibroblasts to synthesize increased quantities of ground substance (glycoproteins and glycosaminoglycans) in which collagen fibers are suspended and move past each other as the skin stretches. Alone, more ground substance will firm up the skin due to the high hygroscopicity and turgidity of hyaluronic acid. Hyaluronic acid is a major component of the ground substance or mucin, in which the fibers are suspended and responsible mainly for retaining water and keeping the dermis hydrated and turgid.
(2) Increasing the proliferative and metabolic activity of fibroblasts, which results in the deposition of new collagen in the upper dermis. Increased collagen adds bulk and density to the skin.
(3) Stimulating blood flow and promoting the formation of vascular tissues (angiogenesis), which improves circulation, enhancing the activity of the other cell types in the dermis.
(4) Thickening of the epidermis, a result of enhanced proliferation of germinative cells, which also contributes to the physical dimension of the surface layer. Increased physical dimension has the effect of adding firmness to the skin.

A firmer, thicker and healthier dermis achieved by applying compounds of formula I in accordance with the present invention inhibits the mobility of easily compressible fat locules, limiting their projection from the subcutaneous fat layer into the overlying dermis.

The present invention also relates to the use of the compounds of the formula I for the preparation of compositions which are suitable for the above-mentioned uses.

The compositions here are usually either compositions which can be used topically, for example cosmetic or dermatological formulations, or foods or food supplements. In this case, the compositions comprise a cosmetically or dermatologically or food-suitable carrier and, depending on the desired property profile, optionally further suitable constituents. In case of pharmaceutical compositions the compositions comprises pharmaceutically acceptable carriers.

Preferred compounds of the formula I are characterised in that R³ is H and R⁴ is OH, since the action potential of representatives of this class of compound is particularly high in the above-mentioned sense. If, in addition, at least one of the radicals R⁵ and R⁶ is OH, these preferred compounds, in addition to the above-mentioned properties, additionally have an antioxidant potential. They can therefore simultaneously function as antioxidant in compositions.

Other preferred compounds of the formula I are characterised in that R⁵ and R⁶ are H. In this case, the radicals R³ and R⁴ are freely accessible, which, as assumed, is advantageous for interaction with enzymes involved in the effects mentioned.

Likewise preferred compounds of the formula I are characterised in that one of the radicals R¹ and R² is H and the other radical is —C(═O)—R⁷, —C(═O)—OR⁷ or a straight-chain or branched C₁- to C₂₀-alkyl group.

Preferably, R⁷ is H or straight-chain or branched C₁- to C₁₅-alkyl group, more preferably H, methyl, ethyl, n-propyl, isopropyl, n-butyl, n-hexyl, 2-ethyl-hexyl, n-nonyl or pentadecyl.

In addition, compounds which are preferred in accordance with the invention have advantages on incorporation into the compositions:
mono- and/or oligoglycosyl radicals improve the water solubility of the compounds to be employed in accordance with the invention;
straight-chain or branched C₁- to C₂₀-alkoxy groups, in particular the long-chain alkoxy functions, such as ethylhexyloxy groups, increase the oil solubility of the compounds;
   i.e. the hydrophilicity or lipophilicity of the compounds according to the invention can be increased through a suitable choice of the substituents.

Glycosidic radicals which can be employed are in particular mono- or oligosaccharide radicals. Preference is given here to hexosyl radicals, in particular ramnosyl radicals and glucosyl radicals. However, other hexosyl radicals, for example allosyl, altrosyl, galactosyl, gulosyl, idosyl, mannosyl and talosyl, may also advantageously be used. It may also be advantageous to use pentosyl radicals. The glycosyl radicals may be linked to the basic structure by means of an α- or β-glycosidic link. A preferred disaccharide is, for example, 6-O-(6-deoxy-α-L-mannopyranosyl)-β-D-glucopyranoside.

However, in likewise preferred embodiments of the invention, the compositions according to the invention may also comprise compounds of the formula I which are sparingly soluble or insoluble in the composition matrix. In this case, the compounds are preferably dispersed in finely divided form.

Further preferred embodiments of the invention, especially combinations, are disclosed in the claims.

Particular preference is given to the use of compounds selected from the compounds of the formulae Ia-In or compounds of the formulae Ia-Ir:

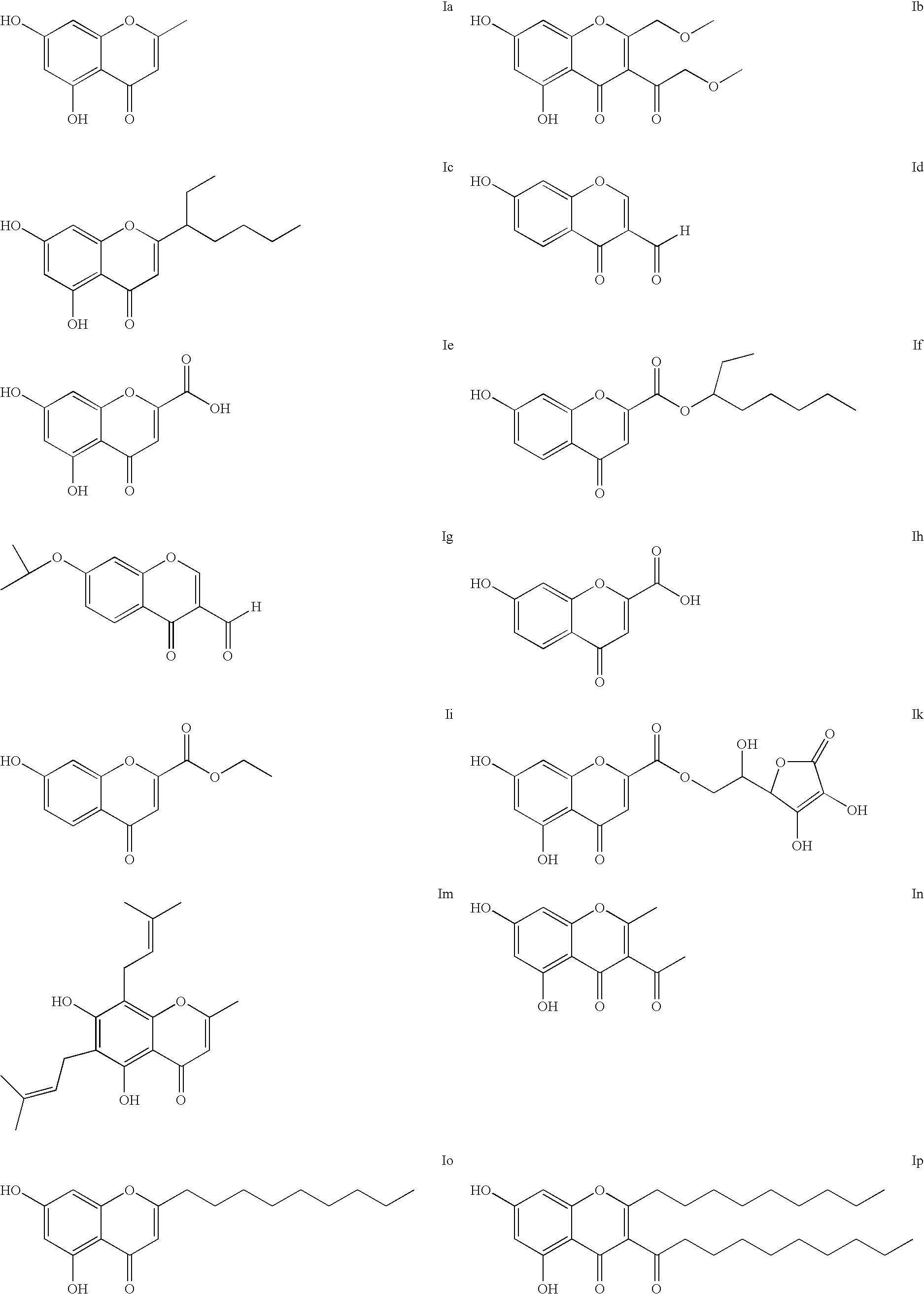

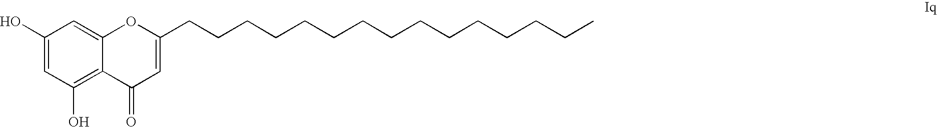

Compound Ia is especially particularly preferred. Compounds Io to Ir are new.

The compounds of the formula I are typically employed in accordance with the invention in amounts of from 0.01 to 20% by weight, preferably in amounts of from 0.025% by weight to 10% by weight and particularly preferably in amounts from 0.5% to 5% by weight and even more preferred in amounts from 0.1% to 1% by weight. The person skilled in the art has absolutely no difficulties in selecting the amount correspondingly depending on the intended action of the composition. How an effective amount of the composition can be determined is discussed below.

The present invention furthermore relates to a composition comprising at least one compound of the formula I containing radicals as defined above, particularly a composition comprising compounds of formulae Io to Ir. The present invention additionally relates to a composition comprising at least one compound of the formula I containing radicals as defined above, particularly a composition comprising compounds of formulae Io to Ir and at least one carrier which is suitable for topical or oral applications.

One skilled in the art will recognize the factors, such as age, weight, general condition of the skin, extent of cellulite and sensitivity to other ingredients, which affect the choice of concentrations used in accordance with the present invention, which may be greater or lower, depending on the individual patient.

By “effective amount” is defined an amount sufficient to provide cellulite reduction or prevention. It is accordingly an object of this invention to provide a composition that can reduce or eliminate cellulite or fat build-ups. Cellulite, as noted above, results from an accumulation of fatty materials and water imprisoned in a matrix made up of more or less watertight compartments. This matrix is comprised of elements of fundamental matter and more particularly of proteoglycons that are polymeric. For oral administration, an effective amount can be achieved by administration of at least about 0.05 mg/day to 20 mg/day, generally at least bout 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day or higher as necessary. Cellulite or fatty response to the dosage can be measured and the dosage modified accordingly. It is recognized that the dose will vary depending upon weight, age, sex, severity of obesity of the patent and the like.

The compositions of the invention can be formulated for oral or topical administration. For oral administration, the composition is administered in a safe and effective dosage for cellulite prevention or reduction and for the treatment of obesity. Oral administration of the composition results in decreased weight gain. Generally, for topical use, the composition is presented in the form of a cream or oil for topical administration, usually in the form of a cream. Thus, the methods of the invention encompass application of the composition used for local slimming and for fighting cellulite.

The composition according to the invention was conceived for fighting conditions of external appearance and figure, such as cellulite, general or local obesity, relaxing or ptosis of the skin and excessive secretion of fat (seborrhoea), which reveal profound bodily dysfunctions. Thus, the compositions of the invention demonstrate a slimming and “rejuvenating” effects on appearance. By using the cream of the invention, good results may be obtained in terms of slimming and of reducing cellulite. That is, the composition is useful for fighting local fat and cellulite. The skin becomes toned and fortified and the user feels no need, from an aesthetic point of view, to use another cream as a supplementing thereof.

The compositions used in the present invention can comprise, consist of, or consist essentially of the essential elements and limitations of the invention described herein, as well any of the additional or optional ingredients, components, or limitations described herein.

The carriers suitable for topical or oral applications according to the invention are carriers well known in the art. Due to the different application fields these carriers can also be called pharmaceutical, cosmetical or dermatological carriers.

In some embodiments, the formulations of the invention comprise a pharmaceutically acceptable carrier. By “pharmaceutically acceptable carrier” is intended a carrier that is conventionally used in the art to facilitate the storage, administration, and/or the healing effect of the therapeutic ingredients. A suitable carrier should be stable, i.e., incapable of reacting with other ingredients in the formulation. It should not produce significant local or systemic adverse effects in recipients at the dosages and concentrations employed for treatment. Such carriers are generally known in the art. Suitable carriers for this invention are those conventionally used large stable macromolecules such as albumin, for example, human serum albumin, gelatin, collagen, polysaccharide, monosaccharides, polyvinyl-pyrrolidone, polylactic acid, polyglycolic acid, polymeric amino acids, fixed oils, ethyl oleate, liposomes, glucose, sucrose, lactose, mannose, dextrose, dextran, cellulose, sorbitol, polyethylene glycol (PEG), and the like. Slow-release carriers, such as hyaluronic acid, may also be suitable. See particularly Prisell et al. (1992) Int. J. Pharmaceu. 85:51-56, and U.S. Pat. No. 5,166,331.

Other acceptable components in the composition include, but are not limited to, pharmaceutically acceptable agents that modify isotonicity including water, salts, sugars, polyols, amino acids, and buffers. Examples of suitable buffers include phosphate, citrate, succinate, acetate, and other organic acids or their salts and salts that modify the tonicity such as sodium chloride, sodium phosphate, sodium sulfate, potassium chloride, and can also include the buffers listed above.

The method for formulating a pharmaceutical composition is generally known in the art. A thorough discussion of formulation and selection of pharmaceutically acceptable carriers, stabilizers, and isomolytes can be found in Remington's Pharmaceutical Sciences (18th ed.; Mack Pub. Co.: Eaton, Pa., 1990), herein incorporated by reference.

In the preferred embodiment of the invention, a cosmetically acceptable vehicle is comprised either of water or of a water/solvent blend. The solvent is optimally chosen from propylene glycol, ethanol, butylene glycol, and polyethylene glycols of various molecular weights.

Vehicles other than water can include liquid or solid emollients, solvents, humectants, thickeners and powders. An especially preferred nonaqueous carrier is a polydimethyl siloxane and/or a polydimethyl phenyl siloxane. Silicones of this invention may be those with viscosities ranging anywhere from about 10 to 10,000,000 centistokes at 25° C.

Especially desirable are mixtures of low and high viscosity silicones. These silicones are available from the General Electric Company under trademarks Vicasil, SE and SF and from the Dow Corning Company under the 200 and 550 Series. Amounts of silicone which can be utilized in the compositions of this invention range anywhere from 5% to 95%, preferably from 25% to 90% by weight of the composition. The cosmetically acceptable vehicle will usually form from 5% to 99.9%, preferably from 25% to 80% by weight of the emulsion, and can, in the absence of other cosmetic adjuncts, form the balance of the composition.

The compositions used in the present invention can also contain a dermatologically acceptable carrier. The phrase “dermatologically-acceptable carrier”, as used herein, means that the carrier is suitable for topical application to the skin, has good aesthetic properties, is compatible with the actives of the present invention and any other components, and will not cause any untoward safety or toxicity concerns.

A safe and effective amount of carrier is from about 50% to about 99.99%, preferably from about 99.9% to about 80%, more preferably from about 98% to about 90%, most preferably from about 95% to 90% of the composition.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following four references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; and U.S. Pat. No. 4,254,105 to Fukuda et al., issued Mar. 3,1981.

The carriers of the skin care compositions can comprise from about 50% to about 99% by weight of the compositions used in the present invention, preferably from about 75% to about 99%, and most preferably from about 85% to about 95%.

Preferred cosmetically and/or pharmaceutically acceptable topical carriers include hydroalcoholic systems and oil-in-water emulsions. When the carrier is a hydro-alcoholic system, the carrier can comprise from about 0% to about 99% of ethanol, isopropanol, or mixtures thereof, and from about 1% to about 99% of water. More preferred is a carrier comprising from about 5% to about 60% of ethanol, isopropanol, or mixtures thereof, and from about 40% to about 95% of water. Especially preferred is a carrier comprising from about 20% to about 50% of ethanol, isopropanol, or mixtures thereof, and from about 50% to about 80% of water. When the carrier is an oil-in-water emulsion, the carrier can include any of the common excipient ingredients for preparing these emulsions. A more detailed discussion of suitable carriers is found in U.S. Pat. No. 5,605,894 to Blank et al., and in PCT application WO 97/39733, published Oct. 30, 1997, to Oblong et al., both herein incorporated by reference in their entirety.

The compositions used in the present invention may optionally comprise additional materials including slimming agents as well as additional actives useful in providing cellulite control. Among these agents are phosphodiesterase inhibitors (e.g., xanthine derivatives such as theophylline, caffeine, theobromine or salts thereof such as aminophylline) and preferred certain oleosoluble vegetable extracts, including, principally, those of climbing ivy (Hedera helix), arnica (Arnica montana), rosemary (Rosmarinus officinalis N), marigold (Calendula officinalis), sage (Salvia officinalis N), ginseng (Panax ginseng), St. Johns-wart (Hypericum perforatum), ruscus (Ruscus aculeatus), meadowsweet (Filipendula ulmaria L) and orthosiphon (Ortosifon stamincus Benth), as well as mixtures of these vegetable extracts, all of which are disclosed in U.S. Pat. No. 4,795,638, herein incorporated by reference.

Also useful are herbal and/or botanical extracts such as those disclosed in U.S. Pat. Nos. 5,705,170 and 5,667,793, both of which are herein incorporated by reference. Mixtures of any of above additional materials may also be used. The compositions used in the present invention may optionally comprise additional skin actives. Non-limiting examples of such skin actives include hydroxy acids such as salicylic acid; desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; corticosteroids such as hydrocortisone, methylprednisolone, dexamethasone, triamcinolone acetconide, and desoxametasone; anesthetics such as benzocaine, dyclonine, lidocaine and tetracaine; antipruitics such as camphor, menthol, oatmeal (colloidal), pramoxine, benzyl alcohol, phenol and resorcinol; antioxidants/radical scavengers such as tocopherol and esters thereof; chelators; hydroxy acids such as glycolic acid; keto acids such as pyruvic acid; N-acetyl-L-cysteine and derivatives thereof; benzofuran derivatives; and skin protectants. Mixtures of any of the above mentioned skin actives may also be used. A more detailed description of these actives is found in U.S. Pat. No. 5, 605,894 to Blank et al. (previously incorporated by reference). Preferred skin actives include hydroxy acids such as salicylic acid, sunscreen, antioxidants and mixtures thereof.

Other conventional skin care product additives may also be included in the compositions used in the present invention. For example, urea, guanidine, glycerol, petrolatum, mineral oil, sugar esters and polyesters, polyolefins, methyl isostearate, ethyl isostearate, cetyl ricinoleate, isononyl isononanoate, isohexadecane, lanolin, lanolin esters, cholesterol, pyrrolidone carboxylic acid/salt (PCA), trimethyl glycine (betaine), tranexamic acid, amino acids (e.g., serine, alanine), panthenol and its derivatives, collagen, hyaluronic acid, elastin, hydrolysates, primrose oil, jojoba oil, epidermal growth factor, soybean saponins, mucopolysaccharides, and mixtures thereof may be used. Other suitable additives or skin actives are discussed in further detail in PCT application WO 97/39733, published Oct. 30, 1997, to Oblong et al., herein incorporated by reference in its entirety.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and hepta-thionine sulfoximine) in very low tolerated doses (for example pmol to μmol/kg), furthermore (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Possible antioxidants are additionally compounds of the general formula I

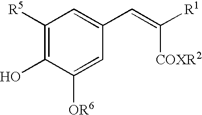

in which
R¹ is selected from the group consisting of —C(O)CH³, —CO₂R³, —C(O)NH₂ and —C(O)N(R⁴)₂,
X is O or NH,
R² is linear or branched alkyl with 1 to 30 C-atoms,
R³ is linear or branched alkyl with 1 to 20 C-atoms,
R⁴ is independently from each other H or linerar or branched alkyl with 1 to 8 C-atoms,
R⁵ is linear or branched alkyl with 1 to 8 C-atoms or linear or branched alkoxy with 1 to 8 C-atoms and
R⁶ is linear or branched alkyl with 1 to 8 C-atoms, preferably derivatives of 2-(4-hydroxy-3,5-dimethoxybenzylidene)-malonic acid, especially preferred 2-(4-hydroxy-3,5-dimethoxybenzylidene)-malonic acid-bis-(2-ethylhexyl)ester (for example Oxynex® ST Liquid).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed with compounds of the formula I in compositions of this type in ratios in the range from 1000:1 to 1:1000, preferably in amounts of from 100:1 to 1:100.

The compositions according to the invention may comprise vitamins as further ingredients. The cosmetic compositions according to the invention preferably comprise vitamins and vitamin derivatives selected from vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin C and derivatives thereof, DL-α-tocopherol, tocopherol E acetate, nicotinic acid, pantothenic acid and biotin. Vitamins are usually employed here with compounds of the formula I in ratios in the range from 1000:1 to 1:1000, preferably in amounts of from 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology & Medicine 2001, 31(7), 869-881, have investigated the pH dependence of the antioxidant action of hydroxyflavones. Quercetin exhibits the greatest activity amongst the structures investigated over the entire pH range.

Suitable antioxidants are furthermore compounds of the formula II

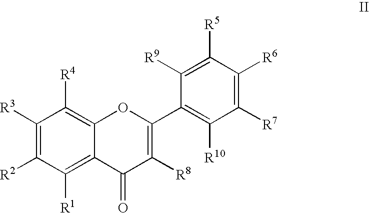

where R¹ to R¹⁰ may be identical or different and are selected from
   H
   OR¹¹
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ— groups, where n=1 to 3,
   where all OR¹¹ are, independently of one another,
      OH
      straight-chain or branched C₁- to C₂₀-alkoxy groups,
      straight-chain or branched C₃- to C₂₀-alkenyloxy groups,
      straight-chain or branched C₁- to C₂₀-hydroxyalkoxy groups, where the hydroxyl group(s) may be bonded to a primary or secondary carbon atom of the chain and furthermore the alkyl chain may also be interrupted by oxygen, and/or
      C₃- to C₁₀-cycloalkoxy groups and/or C₃- to C₁₂-cycloalkenyloxy groups, where the rings may each also be bridged by —(CH₂)ₙ— groups, where n=1 to 3, and/or
      mono- and/or oligoglycosyl radicals,
with the proviso that at least 4 radicals from R¹ to R⁷ are OH and that the molecule contains at least two pairs of adjacent —OH groups,
   or R², R⁵ and R⁶ are OH and the radicals R¹, R³, R⁴ and R⁷⁻¹⁰ are H,
      as described in the German patent application DE-A-10244282.

Compositions which are particularly preferred in accordance with the invention also comprise UV filters besides the compounds of the formula I.

Use of the dibenzoylmethane derivatives, which are particularly preferred as UV-A filters, in combination with the compounds of the formula I gives rise to a further additional advantage: the UV-sensitive dibenzoylmethane derivatives are additionally stabilised by the presence of the compounds of the formula I. The present invention therefore furthermore relates to the use of the compounds of the formula I for the stabilisation of dibenzoylmethane derivatives in compositions.

In principle, all UV filters are suitable for combination with the compounds of the formula I. Particular preference is given to UV filters whose physiological acceptability has already been demonstrated. Both for UVA and UVB filters, there are many proven substances which are known from the specialist literature, for example:
benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292) or isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007) or ethoxylated ethyl 4-aminobenzoate (for example Uvinul® P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
   and further substances, such as
   2-ethylhexyl 2-cyano-3,3-diphenylacrylate (for example Eusolex® OCR),
   3,3′-(1,4-phenylenedimethylene)bis(7,7-dimethyl-2-oxobicyclo[2.2.1]hept-1-ylmethanesulfonic acid and salts thereof (for example Mexoryl® SX),
   2,4,6-trianilino-(p-carbo-2′-ethylhexyl-1′-oxy)-1,3,5-triazine (for example Uvinul® T 150) and
   hexyl 2-(4-diethylamino-2-hydroxybenzoyl)benzoate (for example Uvinul® UVA Plus, BASF).

The compounds mentioned in the list should only be regarded as examples. It is of course also possible to use other UV filters.

These organic UV filters are generally incorporated into cosmetic formulations in an amount of from 0.5 to 10 percent by weight, preferably 1-8%.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]1,3,5-triazine-2,4-diyl)diimino]bis(benzoate) (for example Uvasorb® HEB),
α-(trimethylsilyl)-ω[trimethylsilyl)oxy]poly[oxy(dimethyl [and about 6% of methyl[2-[p-[2,2-bis(ethoxycarbonyl]vinyl]phenoxy]-1-methyleneethyl] and approximately 1.5% of methyl[3-[p-[2,2-bis(ethoxycarbonyl)vinyl]phenoxy]propenyl] and from 0.1 to 0.4% of (methylhydrogen]silylene]] (n≈60) (CAS No. 207 574-74-1)
2,2′-methylenebis(6-(2H-benzotriazol-2-yl)-4-(1,1,3,3-tetramethylbutyl)phenol) (CAS No. 103 597-45-1)
2,2′-(1,4-phenylene)bis(1H-benzimidazole-4,6-disulfonic acid, monosodium salt) (CAS No. 180 898-37-7),
2,4-bis{[4-(2-ethylhexyloxy)-2-hydroxy]phenyl}-6-(4-methoxyphenyl)-1,3,5-triazine (CAS No. 103 597-45-, 187 393-00-6) and
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB).

Further suitable UV filters are also methoxyflavones corresponding to the earlier German patent application DE 10232595.2.

Organic UV filters are generally incorporated into cosmetic formulations in an amount of from 0.5 to 20 percent by weight, preferably 1-15%.

Conceivable inorganic UV filters are those from the group consisting of titanium dioxides, such as, for example, coated titanium dioxide (for example Eusolex® T-2000, Eusolex® T-AQUA), zinc oxides (for example Sachtotec®), iron oxides and also cerium oxides. These inorganic UV filters are generally incorporated into cosmetic compositions in an amount of from 0.5 to 20 percent by weight, preferably 2-10%.

Preferred compounds having UV-filtering properties are 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and its potassium, sodium and triethanolamine salts.

Through combination of one or more compounds of the formula I with further UV filters, the protective action against harmful influences of UV radiation can be optimised.

Optimised compositions may comprise, for example, the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broad-band protection, which can be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables composition problems caused by the interaction of individual composition constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules are preferably present in compositions according to the invention in amounts which ensure that the encapsulated UV filters are present in the composition in the above-indicated amounts.

The skin-protecting or skin-care active ingredients can in principle be any active ingredients known to the person skilled in the art.

In an embodiment of the present invention, particularly preferred active ingredients are pyrimidinecarboxylic acids and/or aryl oximes.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-ups, care creams and sunscreen compositions.

Preference is given here to the use of a pyrimidinecarboxylic acid of the following formula III

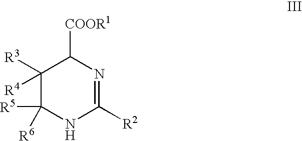

in which R¹ is a radical H or C1-8-alkyl, R² is a radical H or C1-4-alkyl, and R³, R⁴, R⁵ and R⁶ are each, independently of one another, a radical from the group consisting of H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). The compositions according to the invention preferably comprise pyrimidinecarboxylic acids of this type in amounts of up to 15% by weight. The pyrimidinecarboxylic acids are preferably employed here in ratios of from 100:1 to 1:100 with respect to the compounds of the formula I, with ratios in the range from 1:10 to 10:1 being particularly preferred.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which, in addition to the compound of the formula I, additionally comprise an aryl oxime, preferably 2-hydroxy-5-methyllaurophenone oxime, exhibit surprising anti-inflammatory suitability. The compositions here preferably comprise from 0.01 to 10% by weight of the aryl oxime, it being particularly preferred for the composition to comprise from 0.05 to 5% by weight of aryl oxime.

All compounds or components which can be used in the compositions are either known or commercially available or can be synthesised by known processes. The compositions used in the present invention are generally prepared by conventional methods such as are known in the art of making topical compositions. Such methods typically involve mixing of the ingredients in one or more steps to a relatively uniform state, with or without heating, cooling, application of vacuum, and the like. Non-limiting examples of the product form can be a gel, emulsion, lotion, cream, ointment, solution, liquid, etc.

The methods of the present invention are useful for especially preventing cellulite, especially in the subcutaneous, dermis and epidermis tissues of mammalian skin. The methods of the present invention involve topically applying to the skin and effective amount of the skin care composition of the present invention. The amount of the composition which is applied, the frequency of application and the period of use will vary widely depending upon the level of ingredients according to formula I of a given composition and the degree of cellulite fading desired.

The skin care compositions used in the present invention can be chronically applied to the skin. By “chronic topical application” is meant continued topical application of the composition over an extended period during the subject's lifetime, preferably for a period of at least about one week, more preferably for a period of at least about two weeks, even more preferably for a period of at least one month, even more preferably for at least about three months, even more preferably for at least about six months, and more preferably still for at least about one year. While benefits are obtainable after various maximum periods of use (e.g., five, ten or twenty years), it is preferred that chronic application continue throughout the subject's lifetime to maintain and/or increase the benefits achieved. Typically applications would be on the order of one to four times per day over such extended periods, however application rates can be more than four times per day, especially on areas particularly prone to agglomerations of fat and water such as the thighs and buttocks.

The method of treating cellulite is preferably practiced by applying a composition in the form of a skin lotion, cream, gel, cosmetic, or the like which is intended to be left on the skin for some aesthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, most preferably for at least several hours, e.g., up to about 12 hours.

Another approach to ensure a continuous exposure of the skin to at least a minimum level of active ingredient according to the invention is to apply the compound by use of a patch. Such an approach is particularly useful for problem skin areas needing more intensive treatment. The patch can be occlusive, semi-occlusive or non-occlusive. The patch can also include additional actives such as chemical initiators for exothermic reactions such as those described in PCT application WO 9701313 to Burkett et al. Preferably the patch is applied at night as a form of night therapy.

The preferred xanthine optionally employed in the inventive method is caffeine and/or theophylline due to their availability and optimum efficacy. Caffeine and theophylline can be, and preferably are naturally derived, in order to keep with a “natural” character of the inventive compositions. The xanthine is employed in the inventive method preferably in an amount of at least 0.05%, generally in the amount of from 0.05% to 20%, preferably in the amount of from 0.10% to 10%, optimally in the amount of from 0.5% to 3.0% by weight of the composition in order to maximize efficacy at optimum cost.

Another preferred ingredient optionally employed in the inventive method is an alpha hydroxy acid. The presence of the alpha hydroxy acid facilitates the increase in the strength and firmness of dental and epidermal layers of the skin. Even more preferably, the hydroxy acid is chosen from lactic acid, glycolic acid, mandelic acid, and mixtures thereof to optimize the efficacy of compositions by increasing percutaneous absorption. In the most preferred embodiment of the invention, in order to maximize the performance of hydroxy acid, inventive compositions contain the L-form of an alpha hydroxy acid. Preferably the amount of the alpha hydroxy acid component present in the composition according to the invention is from 1.5% to 20%, more preferably from 1.5% to 15%, and most preferably from 3.0% to 12.0% by weight of the composition.

An oil or oily material may be present, together with an emulsifier to provide either a water-in-oil emulsion or an oil-in-water emulsion, depending largely on the average hydrophilic-lipophilic balance (HLB) of the emulsifier employed.

Various types of active ingredients may be employed in the method of the present invention. Actives are defined as skin benefit agents other than emollients and other than ingredients that merely improve the physical characteristics of the composition. Although not limited to this category, general examples include sunscreens, tanning agents, skin anti-wrinkling agents, anti-inflammatory agents, skin lighteners and moisturizers.

Suitable anti-inflammatory compounds include but are not limited to rosmarinic acid, glycyrrizinate derivatives, alpha bisabolol, azulene and derivatives thereof, asiaticoside, sericoside, ruscogenin, escin, esculin, quercetin, rutin, betulinic acid and derivatives thereof, catechin and derivatives thereof.

Suitable vasoactive compounds include but are not limited to papaverine, yohimbine, visnadin, khellin, bebellin, nicotinate derivatives.

Because the stratum coneum is the main barrier to drug penetration, formulations for topical use may include so called skin enhancers such as common solvents, e.g. water, alcokhol, methyl alkyl sulphoxide) or surfactants.

Surfactants, which are also sometimes designated as emulsifiers, may be incorporated into the cosmetic compositions of the present invention as stated above. Surfactants can comprise anywhere from about 0.5% to about 30%, preferably from about 1% to about 15% by weight of the total composition. Surfactants may be cationic, nonionic, anionic, or amphoteric in nature and combinations thereof may be employed.

Illustrative of the nonionic surfactants are alkoxylated compounds based upon fatty alcohols, taffy acids and sorbitan. These materials are available, for instance, from the Shell Chemical Company under the “Neodol” designation. Copolymers of polyoxypropylene- polyoxyethylene, available under the Pluronic trademark sold by the BASF Corporation, are sometimes also useful. Alkyl polyglycosides available from the Henkel Corporation similarly can be utilized for the purposes of this invention.

Anionic-type surfactants may include fatty acid soaps, sodium lauryl sulphate, sodium lauryl ether sulphate, alkyl benzene sulphonate, mono and/or dialkyl phosphates and sodium fatty acyl isethionate.

Amphoteric surfactants include such materials as dialkylamine oxide and various types of betaines (such as cocoamido propyl betaine).

Emollients are often incorporated into cosmetic compositions of the present invention. Levels of such emollients may range from about 0.5% to about 50%, preferably between about 5% and 30% by weight of the total composition. Emollients may be classified under such general chemical categories as esters, fatty acids and alcohols; polyols, hydrocarbons and oils containing at least one amide structural unit.

Some representative oils containing in their structure at least one amide function are especially described with their modes of preparation in EP 1044676 and EP 0928608 from the company Ajinomoto Co. Particularly preferred is isopropyl N-lauroylsarcosinate such as the product marketed under Eldew SL-205 by Ajinomoto.

Esters may be mono- or di-esters. Acceptable examples of fatty di-esters include dibutyl adipate, diethyl sebacate, disopropyl dimerate, and dioctyl succinate. Acceptable branched chain fatty esters include 2-ethyl-hexyl myristate, isopropyl stearate and isostearyl palmitate. Acceptable tribasic acid esters include trisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons that may serve as emollients are those having hydrocarbon chains anywhere from 12 to 30 carbon atoms. Specific examples include aryl alkyl benzoate such as 2-ethylphenyl benzoate, alkyl benzoate, mineral oil, petroleum jelly, squalene and isoparaffins.

Additional emollients or hydrophobic agents include C₁₂ to C₁₅ alkyl benzoate, dioctyl adipate, octyl stearate, octyldodecanol, hexyl laurate, octyldodecyl neopentanoate, cyclomethicone, dicapryl ether, dimethicone, phenyl trimethicone, isopropyl myristate, capriylic/capric glycerides, propylene glycol dicaprylate/dicaprate and decyl oleate.

Another category of functional ingredients within the cosmetic compositions of the present invention are thickeners. A thickener will usually be present in amounts anywhere from 0.1% to 20% by weight, preferably from about 0.5% to 10% by weight of the composition. Exemplary thickeners are cross-linked polyacrylate materials available under the trademark Carbopol from the B. F. Goodrich Company. Gums may be employed such as xanthan, carrageenan, gelatin, karaya, pectin and locust bean gum.

Under certain circumstances the thickening function may be accomplished by a material also serving as a silicone or emollient. For instance, silicone gums in excess of 10 centistokes and esters such as glycerol stearate have dual functionality. Cellulosic derivatives may also be employed, e.g., hydroxypropyl cellulose (Klucel HI®).

Many cosmetic compositions, especially those containing water, must be protected against the growth of potentially harmful microorganisms. Preservatives are, therefore, necessary. Suitable preservatives include alkyl esters of p-hydroxybenzoic acid, hydantoin derivatives, propionate salts, and a variety of quaternary ammonium compounds.

Particularly preferred preservatives of this invention are methyl paraben, propyl paraben, imidazolidinyl urea, sodium dehydroxyacetate and benzyl alcohol. Preservatives will usually be employed in amounts ranging from about 0.5% to 2% by weight of the composition.

Powders may be incorporated into the cosmetic composition employed in the invention. These powders include chalk, talc, Fullers earth, kaolin, starch, smectite clays, chemically modified magnesium aluminum silicate, organically modified montmorillonite clay, hydrated aluminum silicate, filmed silica, aluminum starch octenyl succinate and mixtures thereof.

Other adjunct minor components may also be incorporated into the cosmetic compositions. These ingredients may include coloring agents, opacifiers and perfumes. Amounts of these materials may range anywhere from 0.001% up to 20% by weight of the composition.

The method of the present invention is useful for reducing or preventing the appearance of cellulite, for improving the firmness and elasticity of skin and generally to enhance the quality and flexibility of skin.

The following examples will more fully illustrate the embodiments of this invention, but the invention is not limited thereto. All parts, percentages and proportions referred to herein and in the appended claims are by weight unless otherwise indicated. The following examples further describe and demonstrate embodiments within the scope of the present invention. The examples are given solely for the purpose of illustration and are not to be construed as limitations of the present invention, as many variations thereof are possible without departing from the spirit and scope of the invention.

The invention is explained in greater detail below by means of examples. The invention can be carried out throughout the range claimed and is not restricted to the examples given here.

### EXAMPLES

### Example 1

### Synthesis of 5,7-dihydroxy-2-nonyl-chromen-4-one (1)

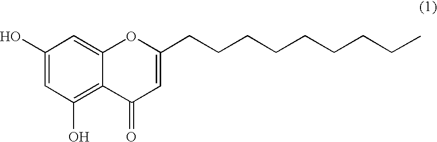

Step 1: Synthesis of 3-decanoyl-5,7-dihydroxy-2-nonyl-chromen-4-one (2).

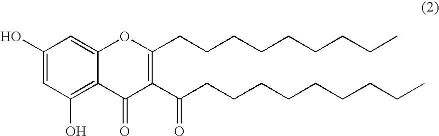

A solution of 2,4,6-trihydroxyacetophenone hydrate (2.0 g, 10.7 mmol) and K₂CO₃ (6.4 g, 44 mmol) in THF (THF=tetrahydrofurane) (40 ml) was heated up to 50° C. in a 100 ml 3-necked round bottom flask fitted with a condenser, a thermometer, a magnetic stirrer and stirred for 15 minutes. A solution of decanoyl chloride (4.43 ml, 21.6 mmol) in THF (40 ml) was added dropwise into the flask at 50° C. The reaction mixture was heated up to reflux for 1 hr. TLC was used to monitor the reaction. After reaction completion, the reaction mixture was poured into ice water. 1N HCl was added until pH value less than 5. A red oil appeared at the top of solution. EEE was used to extract it (EEE=ethyl acetate). The oil was received after evaporation of the EEE phase and recrystallised in mixed solvents (toluene:PE 3:10) to give compound (2); (PE=petrol ether)

¹H NMR (DMSO-d₆)

δ(ppm): 0.886 (t, J=13.5 Hz, 6H, 2-(CH₂)₈—CH₃ and 3-CO—(CH₂)₈—CH₃), ˜1.2-1.45 (m, 28H, 2-CH₂—(CH₂)₇—CH₃ and 3-CO—CH₂—(CH₂)₇—CH₃), 2.633 (t, J=15.5 Hz, 2H, 2-CH₂—(CH₂)₇—CH₃), 2.987 (t, J=14.5 Hz, 2H, 3-CO—CH₂—(CH₂)₇—CH₃), 6.157 (s, 1H, 8-H), 6.253 (s, 1H, 6-H), 9.5 (br, s, 1H, 7-OH), 12.551 (s, 1H, 5-OH).

EI-MS (C₂₈H₄₂O₅): Calculated M⁺=458.630; Found M⁺=458.

Step 2: Synthesis of 5,7-dihydroxy-2-nonyl-chromen-4-one (1) by deprotection of (2).

A suspension of (2) (4.9 g, 10.6 mmol) in 75 ml methanol was heated up to reflux for several minutes. A solution of NaOH (32% solution, 10 ml, 108 mmol) in methanol (25 ml) was dropped slowly into the suspension and reflux was maintained for 1 hr. The reaction mixture was cooled down and some yellow solid precipitated. This mixture was poured into ice water, acidified with HCl until pH was less than 2, and stirred about 30 mins. An organge precipitate formed, was filtered and washed with water to afford the crude product, which was recrystallised in toluene to give the final product (1).

5,7-dihydroxy-2-nonyl-chromen-4-one (1) is a pink powder, m.p.: ˜101° C.

Purity (HPLC analysis): >95%.

¹H NMR (DMSO-d₆):

δ(ppm): 0.865 (t, 3H, 2-(CH₂)₈—CH₃), ˜1.2-1.5 (m, 14H, 2-CH₂-(CH₂)₇—CH₃), 2.571 (t, J=15.5 Hz, 2H, 2-CH₂—(CH₂)₇—CH₃), 5.985 (s, 1H, 3-H), 6.104 (d, J=2 Hz, 1H, 8-H), 6.208 (d, J=2 Hz, 1H, 6-H), 9.382 (s, 1H, 7-OH), 12.803 (s, 1H, 5-OH).

¹³C NMR: (250 MHz, DMSO, Proton decoupled):

δ(ppm): 13.87 (CH₃); 22.02-33.12 (—(CH₂)₈—CH₃); 93.68 (CH); 98.69 (CH); 103.53 (Cquat.); 107.32 (CH); 157.75 (Cquat.); 161.46 (Cquat.); 164.07 (Cquat.); 170.61 (Cquat.); 181.72 (Cquat., C═O).

EI-MS (C₁₈H₂₄O₄): Calculated M⁺=304.381; Found M⁺=304.

### Example 2

### Synthesis and purification method for 5,7-dihydroxy-2-pentadecylchromen-4-one (3).

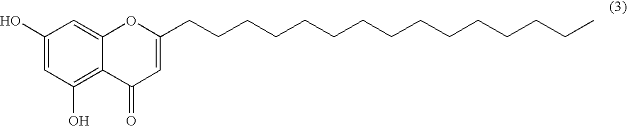

Step 1: Synthesis of 3-hexadecanoyl-5,7-dihydroxy-2-pentadecylchromen-4-one (4).

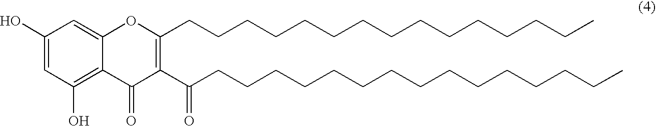

A solution of 2,4,6-trihydroxy acetophenone hydrate (1.0 g, 5.37 mmol) and K₂CO₃ (7.5 g, 54.3 mmol) in THF (15 ml) was heated up to 50° C. under stirring for 15 mins in a 50 ml 3-necked round bottom flask fitted with a condenser and a thermometer. The solution of palmitoyl chloride (6.6 ml, 21.8 mmol) in THF (10 ml) was added dropwise into the flask at 50° C. The reaction mixture was heated up to reflux for 15 mins. TLC was used to monitor the reaction. After completion of the reaction, the mixture was poured into icy water. 1N HCl was added until pH value was smaller than 5. A pink powder appeared, and was collected after filtration was then recrystallised in toluene to give compound (4).

¹H NMR (DMSO-d₆):

δ(ppm): 0.884 (t, J=14 Hz, 6H, 2-(CH₂)₁₄—CH₃ and 3-CO—(CH₂)₁₄—CH₃), ˜1.2-1.45 (m, 52H, 2-CH₂—(CH₂)₁₃—CH₃ and 3-CO—CH₂-(CH₂)₁₃—CH₃), 2.633 (t, J=15.5 Hz, 2H, 2-CH₂—(CH₂)₁₃—CH₃), 2.896 (t, J=14.5 Hz, 2H, 3-CO—CH₂—(CH₂)₁₃—CH₃), 6.155 (s, 1H, 8-H), 6.250 (s, 1H, 6-H), ˜9.6 (br, s, 1H, 7-OH), 12.548 (s, 1H, 5-OH).

EI-MS (C₄₀H₆₆O₅): Calculated M⁺=626.949; Found M⁺=626.

Step 2: Synthesis of 5,7-dihydroxy-2-pentadecyl-chromen-4-one (3) by deprotection of 3-hexadecanoyl-5,7-dihydroxy-2-pentadecyl-chromen-4-one (4)

A suspension of (4) (6.5 g, 10.37 mmol) in 75 ml methanol was heated up to reflux for several mins. A solution of NaOH (32% solution, 10 ml, 108 mmol) in MeOH (25 ml) was dropped slowly into the suspension. Reflux was maintained for 1 hr. The reaction mixture was then cooled down and some yellow solid precipitated. This mixture was poured into ice water, acidified with 1 N HCl until pH was smaller than 2. Stirring was carried on for 30 mins and an orange precipitate appeared. This filter was precipitated and washed with water to afford a crude product, which after recrystallization in toluene gave the final product (3).

5,7-dihydroxy-2-pentadecyl-chromen-4-one (3) is a pink powder, m.p.: ˜108° C.

¹H NMR (DMSO-d₆):

δ(ppm): 0.88 (t, 3H, 2-(CH₂)₁₄—CH₃), ˜1.2-1.5 (m, 26H, 2-CH₂—(CH₂)₁₃—CH₃), 2.571 (t, J=15 Hz, 2H, 2-CH₂—(CH₂)₁₃—CH₃), 5.98 (s, 1H, 3-H), 6.104 (d, J=2 Hz, 1H, 8-H), 6.208 (d, J=2 Hz, 1H, 6-H), 9.372 (s, 1H, 7-OH), 12.803 (s, 1H, 5-OH).

¹³C NMR: (250 MHz, DMSO, Proton decoupled):

δ(ppm): 13.87 (CH₃); 22.02-33.11 (—(CH₂)₁₄—CH₃); 93.68 (CH); 98.70 (CH); 103.51 (Cquat.); 107.31 (CH); 157.74 (Cquat.); 161.46 (Cquat.); 164.11 (Cquat.); 170.57 (Cquat.); 181.72 (Cquat., C═O).

EI-MS (C₂₄H₃₆O₄): Calculated M⁺=388.54; Found M⁺=388.

### Example 3

### Anti-Cellulite Composition

| Ingredients | % |
|---|---|
| Phase A | |
| Cetyl alcohol | 2 |
| Glyceryl Stearate | 5 |
| Caprylic/Capric Triglyceride | 8 |
| Isopropyl Palmitate | 9 |
| Phase B | |
| Glycerin | 3 |
| Preservatives (Germaben II) | 0.8 |
| Water, demineralised | ad 100 |
| 5,7-Dihydroxy-2-Methyl-chromen- | 0.1 |
| 4-one | |

### Procedure:

Heat phases A and B to 65-70° C. Add phase B to phase A without stirring. Homogenize. Cool down to room temperature.

### Notes:

pH value (23° C.): 7.30
Viscosity: 65 000 mPas (Brookfield RVT, spindle C, 5 rpm, Helipath) at 23° C.

### Suppliers:

(1) Merck KGaA/Rona®
(2) Degussa-Goldschmidt AG
(3) Cognis GmbH

### Application:

Apply twice a day, vigorously massaging the LU formulation into skin.

Always carry out circular and up and down motions. Carry out with movements of kneading. If it is well made, the zone becomes red. One should not confine with the thighs, extend this massage to the buttocks and the belly.

Preferably, apply the cream and carry out the massage after a hot shower (not too hot nevertheless!) or a bath. The hydration on the one hand, and the temperature on the other hand, constitute two elements supporting the penetration of the cream through the skin.

Exfoliating as a preliminary step using an exfoliating gel or of a massage glove can also help to prepare the skin well.

### Example 4

### Compositions

Formulations for cosmetic compositions according to the invention are shown by way of example below. The INCI names of the commercially available compounds are also shown.

UV-Pearl, OMC stands for the composition having the INCI name: Water (for EU: Aqua), Ethylhexyl Methoxycinnamate, Silica, PVP, Chlorphenesin, BHT; this composition is commercially available under the name Eusolex®UV Pearl™ OMC from Merck KGaA, Darmstadt.

The other UV Pearl products indicated in the tables are each of analogous composition with OMC replaced by the UV filter indicated.

TABLE 1
W/O emulsions (data in % by weight)

| | 1-1 | 1-2 | 1-3 | 1-4 | 1-5 | 1-6 | 1-7 | 1-8 | 1-9 | 1-10 |
|---|---|---|---|---|---|---|---|---|---|---|
| Titanium Dioxide | | 2 | 5 | | | | | | | 3 |
| 2-Methyl-5,7-dihydroxy- | 5 | 3 | 2 | 1 | 2 | | | | 1 | 1 |
| chromen-4-one | | | | | | | | | | |
| 2-(1-Ethylhexyl)-5,7- | | | | | | 1 | 2 | 1 | | |
| dihydroxychromen-4-one | | | | | | | | | | |
| Zinc Oxide | | | | | | | | 5 | 2 | |
| UV-Pearl, OMC | 30 | 15 | 15 | 15 | 15 | 15 | 15 | 15 | 15 | 15 |
| Polyglyceryl-3-Dimerate | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 |
| caffeine | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Water | to 100 | to 100 | to 100 | to 100 | to 100 | to 100 | to 100 | to 100 | to 100 | to 100 |

| | 1-11 | 1-12 | 1-13 | 1-14 | 1-15 | 1-16 | 1-17 | 1-18 |
|---|---|---|---|---|---|---|---|---|
| Titanium Dioxide | 3 | | 2 | | 3 | | 2 | 5 |
| Benzylidene Malonate Polysiloxane | | 1 | 0.5 | | | | | |
| Methylene Bis-benzotriazolyl | 1 | 1 | 0.5 | | | | | |
| Tetramethylbutylphenol | | | | | | | | |
| 2-(1-Ethylhexyl)-5,7-dihydroxy- | 5 | 3 | 2 | 5 | 1 | 3 | 7 | 2 |
| chromen-4-one | | | | | | | | |
| Polyglyceryl-3-Dimerate | 3 | 3 | 3 | 3 | | | | |
| Cera Alba | 0.3 | 0.3 | 0.3 | 0.3 | 2 | 2 | 2 | 2 |
| Hydrogenated Castor Oil | 0.2 | 0.2 | 0.2 | 0.2 | | | | |
| Paraffinium Liquidum | 7 | 7 | 7 | 7 | | | | |
| Caprylic/Capric Triglyceride | 7 | 7 | 7 | 7 | | | | |
| Hexyl Laurate | 4 | 4 | 4 | 4 | | | | |
| PVP/Eicosene Copolymer | 2 | 2 | 2 | 2 | | | | |
| Propylene Glycol | 4 | 4 | 4 | 4 | | | | |
| Magnesium Sulfate | 0.6 | 0.6 | 0.6 | 0.6 | | | | |
| Caffeine | 0.5 | 0.5 | 0.5 | 0.5 | | | | |
| Tocopheryl Acetate | 0.5 | 0.5 | 0.5 | 0.5 | 1 | 1 | 1 | 1 |
| Cyclomethicone | 0.5 | 0.5 | 0.5 | 0.5 | | | | |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Dicocoyl Pentyerythrityl Citrate (and) | | | | | 6 | 6 | 6 | 6 |
| Sorbitan Sesquioleate (and) Cera | | | | | | | | |
| Alba (and) Aluminium Stearate | | | | | | | | |
| PEG-7 Hydrogenated Castor Oil | | | | | 1 | 1 | 1 | 1 |
| Zinc Stearate | | | | | 2 | 2 | 2 | 2 |
| Oleyl Erucate | | | | | 6 | 6 | 6 | 6 |
| Decyl Oleate | | | | | 6 | 6 | 6 | 6 |
| Dimethicone | | | | | 5 | 5 | 5 | 5 |
| Tromethamine | | | | | 1 | 1 | 1 | 1 |
| Glycerine | | | | | 5 | 5 | 5 | 5 |
| Allantoin | | | | | 0.2 | 0.2 | 0.2 | 0.2 |
| Water | to 100 | to 100 | to 100 | to 100 | to 100 | to 100 | to 100 | to 100 |

| | 1-19 | 1-20 | 1-21 | 1-22 | 1-23 | 1-24 | 1-25 | 1-26 | 1-27 | 1-28 | 1-29 |
|---|---|---|---|---|---|---|---|---|---|---|---|
| Titanium Dioxide | | 2 | 5 | | | | | | | 3 | 3 |
| Benzylidene Malonate Polysiloxane | | | | 1 | | | | | 1 | 1 | |
| Zinc Oxide | | | | | | | | 5 | 2 | | |
| 2-Methyl-5,7-dihydroxychromen-4- | 5 | 5 | 5 | 5 | 7 | 5 | 5 | 5 | 5 | 5 | 8 |
| one | | | | | | | | | | | |
| UV-Pearl, OCR | | 10 | | | | | | | | | 5 |
| UV-Pearl, EthylhexylDimethylPABA | | | 10 | | | | | | | | |
| UV-Pearl, Homosalate | | | | 10 | | | | | | | |
| UV-Pearl, Ethylhexyl Salicylate | | | | | 10 | | | | | | |
| UV-Pearl, OMC. BP-3 | | | | | | 10 | | | | | |
| UV-Pearl, OCR. BP-3 | | | | | | | 10 | | | | |
| UV-Pearl, Ethylhexyl Dimethyl | | | | | | | | 10 | | | |
| PABA, BP-3 | | | | | | | | | | | |
| UV-Pearl, Homosalate, BP-3 | | | | | | | | | 10 | | |
| UV-Pearl, Ethylhexyl Salicylate, | | | | | | | | | | 10 | |
| BP-3 | | | | | | | | | | | |
| BMDBM | | | | | | | | | | | 2 |
| UV-Pearl, OMC, | 25 | | | | | | | | | | |
| 4-Methylbenzylidene Camphor | | | | | | | | | | | |
| Polyglyceryl-3-Dimerate | 3 | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 | 0.6 |
| Theophylline | 0.5 | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 | 0.15 |

| | |
|---|---|
| Water | to 100 |

TABLE 2
O/W emulsions, data in % by weight

| | 2-1 | 2-2 | 2-3 | 2-4 | 2-5 | 2-6 | 2-7 | 2-8 | 2-9 | 2-10 |
|---|---|---|---|---|---|---|---|---|---|---|
| Titanium Dioxide | | 2 | 5 | | | | | | | 3 |
| Methylene Bis-benzotriazolyl | | | | | | 1 | 2 | 1 | | |
| Tetramethylbutylphenol | | | | | | | | | | |
| 2-(1-Ethylhexyl)-5,7-dihydroxy- | | | | 1 | 2 | | | | 1 | 1 |
| chromen-4-one | | | | | | | | | | |
| 4′-Methoxy-6-hydroxyflavone | 1 | 3 | | 2 | | 5 | | 5 | 2 | |
| 2-(Methoxy-methyl)-5,7- | 5 | 5 | 5 | 5 | 5 | 5 | 5 | 5 | 5 | 5 |
| dihydroxychromen-4-one | | | | | | | | | | |
| 2-Carboxy-5,7-dihydroxy- | 1 | 5 | 4 | | 6 | | 7 | | 2 | 1 |
| chromen-4-one | | | | | | | | | | |
| 4-Methylbenzylidene Camphor | 2 | | 3 | | 4 | | 3 | | 2 | |
| BMDBM | 1 | 3 | | 3 | 3 | | 3 | 3 | 3 | |
| Stearyl Alcohol (and) Steareth-7 | 3 | 3 | 3 | 3 | 3 | 3 | 3 | 3 | 3 | 3 |
| (and) Steareth-10 | | | | | | | | | | |
| Glyceryl Stearate (and) Ceteth- | 3 | 3 | 3 | 3 | 3 | 3 | 3 | 3 | 3 | 3 |
| 20 | | | | | | | | | | |
| Glyceryl Stearate | 3 | 3 | 3 | 3 | 3 | 3 | 3 | 3 | 3 | 3 |
| Microwax | 1 | 1 | 1 | 1 | 1 | 1 | 1 | 1 | 1 | 1 |
| Cetearyl Octanoate | 11.5 | 11.5 | 11.5 | 11.5 | 11.5 | 11.5 | 11.5 | 11.5 | 11.5 | 11.5 |
| Caprylic/Capric Triglyceride | 6 | 6 | 6 | 6 | 6 | 6 | 6 | 6 | 6 | 6 |
| Oleyl Oleate | 6 | 6 | 6 | 6 | 6 | 6 | 6 | 6 | 6 | 6 |
| Propylene Glycol | 4 | 4 | 4 | 4 | 4 | 4 | 4 | 4 | 4 | 4 |
| Glyceryl Stearate SE | | | | | | | | | | |
| Stearic Acid | | | | | | | | | | |
| Persea Gratissima | | | | | | | | | | |
| Propylparaben | 0.05 | 0.05 | 0.05 | 0.05 | 0.05 | 0.05 | 0.05 | 0.05 | 0.05 | 0.05 |
| Caffeine | 0.15 | 0.15 | 0.15 | 0.15 | 0.15 | 0.15 | 0.15 | 0.15 | 0.15 | 0.15 |
| Tromethamine | | | 1.8 | | | | | | | |
| Glycerine | | | | | | | | | | |
| Water | to 100 | to 100 | to 100 | to 100 | to 100 | to 100 | to 100 | to 100 | to 100 | to 100 |

| | 2-11 | 2-12 | 2-13 | 2-14 | 2-15 | 2-16 | 2-17 | 2-18 |
|---|---|---|---|---|---|---|---|---|
| Titanium Dioxide | 3 | | 2 | | | | 2 | 5 |
| Benzylidene Malonate Polysiloxane | | 1 | 0.5 | | | | | |
| Methylene Bis-benzotriazolyl | 1 | 1 | 0.5 | | | | | |
| Tetramethylbutylphenol | | | | | | | | |
| 4′-Methoxy-7-(β-glucoside Flavone | | | | 1 | 2 | | | |
| 2-Carboxyl-5,7-dihydroxychromen- | 1 | 3 | | 2 | | 5 | | 5 |
| 4-one | | | | | | | | |
| 2-Carboxy-7-hydroxychromen-4- | 5 | 5 | 5 | 5 | 5 | 5 | 5 | 5 |
| one | | | | | | | | |
| Ethyl 5,7-Dihydroxychromen-4- | 1 | 5 | 4 | | 6 | | 7 | |
| one-2-carboxylate | | | | | | | | |
| Zinc Oxide | | | 2 | | | | | |
| UV-Pearl, OMC | 15 | 15 | 15 | 30 | 30 | 30 | 15 | 15 |
| 4-Methylbenzylidene Camphor | | | | 3 | | | | |
| BMDBM | | | | 1 | | | | |
| Phenylbenzimidazole Sulfonic Acid | | | | | 4 | | | |
| Stearyl Alcohol (and) Steareth-7 | 3 | 3 | 3 | 3 | | | | |
| (and) Steareth-10 | | | | | | | | |
| Glyceryl Stearate (and) Ceteth-20 | 3 | 3 | 3 | 3 | | | | |
| Glyceryl Stearate | 3 | 3 | 3 | 3 | | | | |
| Microwax | 1 | 1 | 1 | 1 | | | | |
| Cetearyl Octanoate | 11.5 | 11.5 | 11.5 | 11.5 | | | | |
| Caprylic/Capric Triglyceride | 6 | 6 | 6 | 6 | 14 | 14 | 14 | 14 |
| Oleyl Oleate | 6 | 6 | 6 | 6 | | | | |
| Propylene Glycol | 4 | 4 | 4 | 4 | | | | |
| Glyceryl Stearate SE | | | | | 6 | 6 | 6 | 6 |
| Stearic Acid | | | | | 2 | 2 | 2 | 2 |
| Persea Gratissima | | | | | 8 | 8 | 8 | 8 |
| Propylparaben | 0.05 | 0.05 | 0.05 | 0.05 | 0.05 | 0.05 | 0.05 | 0.05 |
| Theophylline | 0.15 | 0.15 | 0.15 | 0.15 | 0.15 | 0.15 | 0.15 | 0.15 |
| Tromethamine | | | | | 1.8 | | | |
| Glycerine | | | | | 3 | 3 | 3 | 3 |
| Water | to 100 | to 100 | to 100 | to 100 | to 100 | to 100 | to 100 | to 100 |

| | 2-19 | 2-20 | 2-21 | 2-22 | 2-23 | 2-24 | 2-25 | 2-26 | 2-27 | 2-28 |
|---|---|---|---|---|---|---|---|---|---|---|
| Titanium Dioxide | | | | | | | 3 | 3 | | 2 |
| Benzylidene Malonate | 1 | 2 | | | | 1 | 1 | | 1 | 0.5 |
| Polysiloxane | | | | | | | | | | |
| 7,8,3′,4′-Tetrahydroxyflavone | | | | 1 | 2 | | | | 1 | 1 |
| Ethyl 5,7-Dihydroxychromen-4- | 1 | 3 | | 2 | | 5 | | 5 | 2 | |
| on-2-carboxylate | | | | | | | | | | |
| 2-Methyl-5,7-dihydroxy- | 5 | 5 | 5 | 5 | 5 | 5 | 5 | 5 | 5 | 5 |
| chromen-4-one | | | | | | | | | | |
| Methylene Bis-benzotriazolyl | | | 1 | 2 | 1 | | | 1 | 1 | 0.5 |
| Tetramethylbutylphenol | | | | | | | | | | |
| Zinc Oxide | | | | | 5 | 2 | | | | 2 |
| UV-Pearl, OMC | 15 | 15 | 15 | 15 | 15 | 15 | 15 | 15 | 15 | 15 |
| Caprylic/Capric Triglyceride | 14 | 14 | 14 | 14 | 14 | 14 | 14 | 14 | 14 | 14 |
| Oleyl Oleate | | | | | | | | | | |
| Propylene Glycol | | | | | | | | | | |
| Glyceryl Stearate SE | 6 | 6 | 6 | 6 | 6 | 6 | 6 | 6 | 6 | 6 |
| Stearic Acid | 2 | 2 | 2 | 2 | 2 | 2 | 2 | 2 | 2 | 2 |
| Persea Gratissima | 8 | 8 | 8 | 8 | 8 | 8 | 8 | 8 | 8 | 8 |
| Theophylline | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Glycerine | 3 | 3 | 3 | 3 | 3 | 3 | 3 | 3 | 3 | 3 |
| Water | to 100 | to 100 | to 100 | to 100 | to 100 | to 100 | to 100 | to 100 | to 100 | to 100 |

TABLE 3
Gels, data in % by weight

| | 3-1 | 3-2 | 3-3 | 3-4 | 3-5 | 3-6 | 3-7 | 3-8 | 3-9 | 3-10 |
|---|---|---|---|---|---|---|---|---|---|---|
| a = aqueous gel | | | | | | | | | | |
| Titanium Dioxide | | 2 | 5 | | | | | | | 3 |
| 2-Methyl-5,7-Dihydroxychromen-4- | | | | 1 | 2 | | | | 1 | 1 |
| one | | | | | | | | | | |
| Ethyl 5,7-Dihydroxy-chromen-4- | 1 | 3 | | 2 | | 5 | | 5 | 2 | |
| one-2-carboxylate | | | | | | | | | | |
| Benzylidene Malonate Polysiloxane | | | 1 | 1 | 2 | | | | 1 | 1 |
| Methylene Bis-benzotriazolyl | | 1 | | | | 1 | 2 | 1 | | |
| Tetramethylbutylphenol | | | | | | | | | | |
| Zinc Oxide | | | | 2 | | | | 5 | 2 | |
| UV-Pearl, Ethylhexyl | 30 | 15 | 15 | 15 | 15 | 15 | 15 | 15 | 15 | 15 |
| Methoxycinnamate | | | | | | | | | | |
| 4-Methylbenzylidene Camphor | | | | | 2 | | | | | |
| Butylmethoxydibenzoylmethane | | 1 | | | | | | | | |
| Phenylbenzimidazole Sulfonic Acid | | | 4 | | | | | | | |
| Prunus Dulcis | 5 | 5 | 5 | 5 | 5 | 5 | 5 | 5 | 5 | 5 |
| Tocopheryl Acetate | 0.5 | 0.5 | 0.5 | 0.5 | 0.5 | 0.5 | 0.5 | 0.5 | 0.5 | 0.5 |
| Caprylic/Capric Triglyceride | 3 | 3 | 3 | 3 | 3 | 3 | 3 | 3 | 3 | 3 |
| Octyldodecanol | 2 | 2 | 2 | 2 | 2 | 2 | 2 | 2 | 2 | 2 |
| Decyl Oleate | 2 | 2 | 2 | 2 | 2 | 2 | 2 | 2 | 2 | 2 |
| PEG-8 (and) Tocopherol (and) | 0.05 | 0.05 | 0.05 | 0.05 | 0.05 | 0.05 | 0.05 | 0.05 | 0.05 | 0.05 |
| Ascorbyl Palmitate (and) Ascorbic | | | | | | | | | | |
| Acid (and) Citric Acid | | | | | | | | | | |
| Sorbitol | 4 | 4 | 4 | 4 | 4 | 4 | 4 | 4 | 4 | 4 |
| Polyacrylamide (and) C13-14 | 3 | 3 | 3 | 3 | 3 | 3 | 3 | 3 | 3 | 3 |
| Isoparaffin (and) Laureth-7 | | | | | | | | | | |
| Propylparaben | 0.05 | 0.05 | 0.05 | 0.05 | 0.05 | 0.05 | 0.05 | 0.05 | 0.05 | 0.05 |
| Caffeine | 0.15 | 0.15 | 0.15 | 0.15 | 0.15 | 0.15 | 0.15 | 0.15 | 0.15 | 0.15 |
| Tromethamine | | | 1.8 | | | | | | | |
| Water | to 100 | to 100 | to 100 | to 100 | to 100 | to 100 | to 100 | to 100 | to 100 | to 100 |

1. A method for nontherapeutically preventing, reducing or combating signs of cellulite or cellulite and/or reducing localized fatty excesses comprising employing least one compound of the formula I 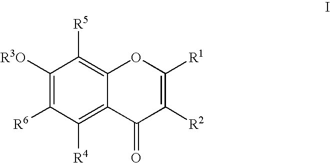
or of a composition comprising at least one compound of the formula I, where
R¹ and R² may be identical or different and are selected from
H, —C(═O)—R⁷ and —C(═O)—OR⁷,
straight-chain or branched C₁- to C₂₀-alkyl groups,
straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,
R³ is H or straight-chain or branched C₁- to C₂₀-alkyl groups,
R⁴ is H or OR⁸,
R⁵ and R⁶ may be identical or different and are selected from
—H and —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ is H, straight-chain or branched C₁- to C₂₀-alkyl groups, a polyhydroxyl compound, such as, preferably, an ascorbic acid radical or glycosidic radicals, and
R⁸ is H or straight-chain or branched C₁- to C₂₀-alkyl groups,
where at least two of the substituents R¹, R² and R⁴-R⁶ are different from H or at least one substituent from R¹ and R² is —C(═O)—R⁷ or —C(═O)—OR⁷.

2. A method of claim 1, wherein R³ is H, and R⁴ is OH, where at least one of the radicals R⁵ and R⁶ is preferably additionally OH.

3. A method of claim 1 wherein R⁵ and R⁶ are H.

4. A method of claim 1 wherein according to one of the radicals R¹ and R² is H and the other radical is —C(═O)—R⁷, —C(═O)OR⁷ or a straight-chain or branched C₁- to C₂₀-alkyl group.

5. A method of claim 1 wherein the compound of the formula I is a compound selected from the compounds of the formulae Ia-Ir:

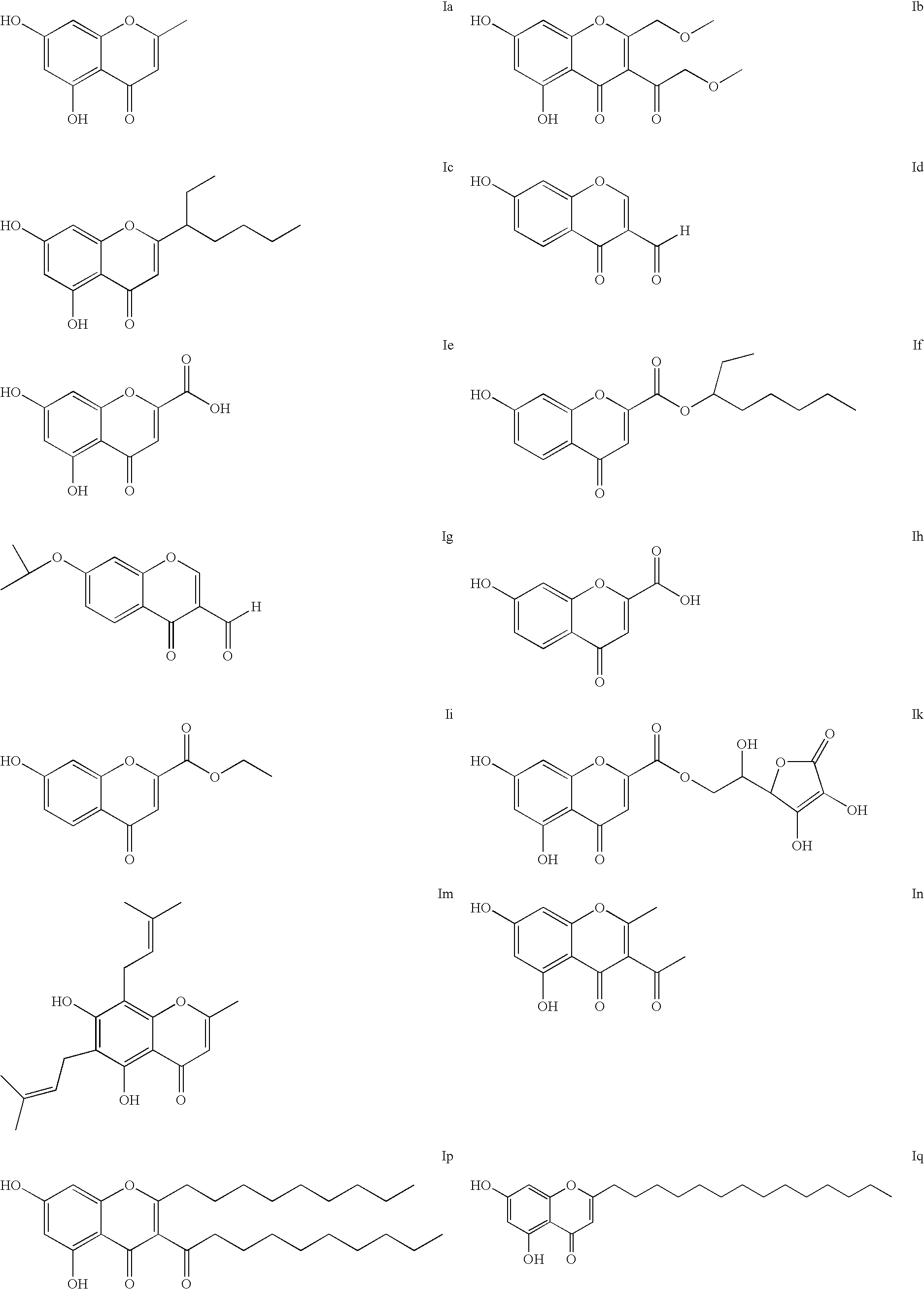

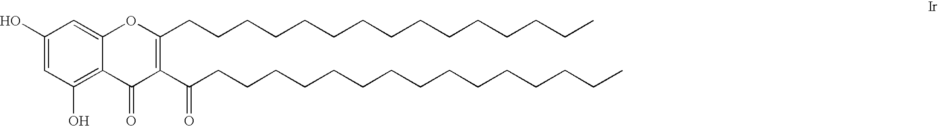

6. Composition comprising at least one compound of the formula I containing radicals as defined in claim 1, at least one carrier which is suitable for topical or oral applications.

7. Composition according to claim 6, characterised in that the composition comprises one or more compounds of formulae Io to Ir.

8. Composition according to claim 6, characterised in that the compounds of formula I are present in an amount of from 0.01 to 20% by weight.

9. Composition according to claim 6, where the composition comprises one or more UV filters, which are preferably selected from the group consisting of 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and its potassium, sodium and triethanolamine salts.

10. Composition according to claim 6, characterised in that the composition further comprises an additional active, which are selected from the group consisting of phosphodiesterase inhibitors, oleosoluble vegetable extracts, herbal extracts, botanical extracts or mixtures thereof.

11. Process for the preparation of a composition according to claim 6, characterised in that at least one compound of the formula I containing radicals as defined in one or more of claims 1 to 5 is mixed with a carrier for topical or oral applications.

12. Process for the preparation of a composition according to claim 11, characterised in that the carrier for topical or oral applications is a cosmetically or dermatologically or pharmaceutically carrier.

13. Use of at least one compound of the formula I 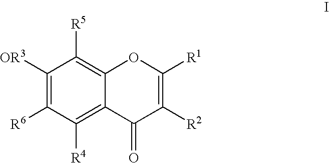
where
R¹ and R² may be identical or different and are selected from
H, —C(═O)—R⁷ and —C(═O)—OR⁷,
straight-chain or branched C₁- to C₂₀-alkyl groups,
straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,
R³ is H or straight-chain or branched C₁- to C₂₀-alkyl groups,
R⁴ is H or OR⁸,
R⁵ and R⁶ may be identical or different and are selected from
—H and —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ is H, straight-chain or branched C₁- to C₂₀-alkyl groups, a polyhydroxyl compound, such as, preferably, an ascorbic acid radical or glycosidic radicals, and
R⁸ is H or straight-chain or branched C₁- to C₂₀-alkyl groups,
where at least two of the substituents R¹, R² and R⁴-R⁶ are different from H or at least one substituent from R¹ and R² is —C(═O)—R⁷ or —C(═O)—OR⁷, for the preparation of a pharmaceutical composition for the prevention of, reduction of or to combat signs of cellulite or cellulite and/or for the reduction of localized fatty excesses.
